(19) United States
(12) Patent Application Publication
(10) Pub. No.: US 2008/0050436 A1
(43) Pub. Date: Feb. 28, 2008

(54) METHODS AND COMPOUNDS FOR OBLITERATION OF VESSELS

(76) Inventor: Jack F. Chu, Santa Rosa, CA (US)

Correspondence Address: JACK CHU, 5918 SUNHAWK DRIVE, SANTA ROSA, CA, 95409, US

(21) Appl. No.: 11/467,530

(22) Filed: Aug. 25, 2006

(51) Int. Cl. A61K 31/60; A61K 33/26; A61K 9/14

(52) U.S. Cl. 424/488; 514/159; 424/646

(57) ABSTRACT

A minimally invasive method that allows for complete obliteration of the affected vessels without scarring or any of the other undesirable complications of conventional or foam sclerotherapy. More particularly, the present invention relates to a method for using a non-foaming thickener to reduce dilution and diffusion of the sclerosant in the blood vessel and enhance the efficacy of the sclerotherapy treatment. The thickener can be thickening agent, hydrogel, environmentally sensitive hydrogel, and self-assembly polymer, etc. After it is mixed with sclerosant and injected into the blood vessel through a needle or a catheter, the compound will replace blood and obliterate the affected vessels.

### FIELD OF THE INVENTION

This invention relates to new treatment of varicose veins, hemorrhoids, venous and coronary insufficiencies, esophageal varices, venous-drainage-impotence of the penis, vascular malformation, and excessive blood supplied to tumors. It is a minimally invasive method that allows for complete obliteration of the affected vessels without scarring or any of the other undesirable complications of traditional or foams sclerotherapy. More particularly, the present invention relates to a method for using a non-foaming thickener to reduce dilution and diffusion of the sclerosant in the blood vessel and enhance the efficacy of the sclerotherapy treatment.

### BACKGROUND OF THE INVENTION

Varicose vein of the leg encompass the most frequent physical signs of chronic venous insufficiency (CVI) and is one of the most prevalent conditions in patients with venous diseases. The term “varicose veins” is used to describe dilated blood vessels under the skin of the legs, which has become visible, twisted, tortuous, abnormally enlarged or elongated. It is a medical condition present in up to 25% of the adult population in the developed countries and is especially prevalent among female population. The estimated prevalence rate of severe varicose veins of lower extremities is 2.8% of the U.S. population (7.4 million cases). Over 50% of the population over 70 has varicose veins. The estimated treatment cost is up to $2.5 billion annually.

There is no known cure for varicose veins. Risk factors for varicose veins include genetic predisposition, hormonal changes, pregnancy, obesity, lifestyle, venous thrombosis, leg injury, and prolonged standing. It is caused either by abnormalities of the venous wall (dilation of the wall) and/or valve leaflets or as a consequence of previous thrombosis. These one-way valves within the veins provide an important function in controlling blood pressure within the venous system of the legs. During exercising, the leg muscles provide a muscular pump that compresses the veins and propels blood to the heart. Efficiency of the muscular pump is enhanced by the one-way valves within the veins that protect the venous system at the lower extremities from excess pressure generated by gravity, coughing, straining, lifting, or standing. The failure of the valves can lead to reflux, excess pressure at the limbs and eventually varicose veins.

Leg varicosities can range from minor dilations to large structures in the calf. Small varicose veins (<2 mm in diameter) are referred to as venulectasia, telangiectasia, thread veins, spider veins, matted veins or dermal flares, and often occur alongside large varicose veins. Large varicose veins (>2 mm in diameter) include varicosities of reticular and saphenous veins and/or varicosities of their larger tributaries. They also include varicose veins deep below the skin surface. Both prevalence and incidence increase with age in both genders.

Patients suffering from varicose veins often experience various symptoms, including aching, itching, burning sensation, heaviness, swelling, restless, skin changes, edema, or cramping of the legs. More serious complications of the varicose veins can include thrombophlebitis, dermatitis, hemorrhage and ulcers. The symptoms are not always related to the degree or size of the varicose veins. However, varicose veins do not usually cause severe complications even if they are present over very long period of time. Many patients seek medical treatment of varicose veins for primarily cosmetic reasons due to the generally unsightly appearance that characterizes the condition. One survey showed that 18% of persons with varicose veins have continuous symptoms.

Currently, several treatment options are available depending upon the pathology and the severity of the condition. Conservative measures, such as compression therapy, are suitable for small varicose veins with mild symptoms and no severe complications. However, the treatment is not curative, and patients usually have to wear compression stockings for life. When conservative measures fail and for more complicated cases, the main options are sclerotherapy, surgery, laser therapy, and radio frequency therapy. Many patients are treated using a combination of surgery, sclerotherapy and compression therapy. Each therapy has its advantages and disadvantages.

Surgical removal of the superficial varicose veins is also called “vein stripping”. Once the veins are completely removed from the leg, the various incision wounds can be sutured closed. The stripping technique has several significant drawbacks. The numerous incisions alone the veins often leave substantial scars on the legs. There are also complications such as blood loss, pain, infection, hematoma, nerve injury and swelling. After the procedure, a patient usually requires several weeks to recover. As a result of those drawbacks, the surgery is usually recommended only for patients with extreme cases of varicose veins.

Several catheter based treatment systems (abrasion, radio frequency, laser, microwave, radio frequency energy, etc.) have been developed to treat varicose veins in the past several years. However, the major disadvantage for those systems is the cost and complexity of the catheter and the systems. The treatment is usually slow and painful. The patient must be sufficiently anaesthetized along the entire length of the veins to be treated. Re-position the catheter in the veins is also time-consuming. Furthermore, tributary veins remain unaffected and must be treated separately. In addition, even with some promising early results from radio frequency (RF) and laser therapies, the long-term durability of those treatments is still in question. Both RF and Laser therapies leave a proximal section of great saphenous vein (GSV) untreated to avoid the deep vein thrombosis. The proximal GSV tributaries are also difficult to reach by catheters and have to be treated with other methods such as stripping or sclerotherapy.

Sclerotherapy has been used to treat all types and sizes of varicose veins with varying results. It has been advocated and used as an alternative to surgery or in combination with surgery for treating varicose veins. The principle of successful sclerotherapy is to cause irreversible endothelial injury in the target vein and eliminate reflux without causing any adverse effects. The procedure involves direct injection of a corrosive substance (sclerosing agent or sclerosant) into the varicose veins with a needle. The sclerosant is designed to irritate, dehydrate, change the surface tension of or destroy the endothelial cells. This causes a localized inflammatory reaction and produces small thrombosis that eventually results in permanent fibrosis and elimination of the vein. After that, compression therapy may be applied to assist healing for a few days or as long as 8 weeks. The sclerotherapy is often combined with an operative procedure, such as stripping, ligation of a portion of the saphenous vein or other catheter based therapies. Patients require no hospitalization and remain mobile and active after sclerotherapy. Major complications are very rare and include deep vein thrombosis, superficial thrombophlebitis, pulmonary embolism, and systemic allergic reaction.

The issues with sclerotherapy are that it has a high rate of recurrence and usually can't be used for larger vein (i.e. saphenous veins) in the upper thigh region due to the risk of sclerosis of the deep veins. Treatment accuracy is increased significantly with the use of ultrasound or Doppler guided injections and endoscopic injection procedures. The success can be as high as 80% when the sclerotherapy is combined with good surgical technique. However, success rate in the range of 60% is more common.

Various practices in sclerotherapy have been done by many clinicians. Techniques vary in terms of patient position, sclerosant injection quantity, volume, and concentration injected at treatment sites. Those factors have been identified as potential aspects that can influence the therapeutic outcome. Study by Lee, et al (2003) indicates that acute morbidity or toxicity of absolute ethanol sclerotherapy is directly proportional to the total amount of ethanol injected. Iwamoto et al. found that the thrombotic complications rate was significantly higher in patients with a higher total volume of sclerosant (6.8+/−6.2 ml hypertonic saline) used than that in patients with lower volume (3.4+/−5.7 ml) in 198 limbs. As a result, they recommended using small amount of sclerosing agents if possible. Due to the concerns of using too much sclerosant at one treatment, sclerotherapy generally requires multiple treatment sessions at intervals. It may take up to 6 months, depending on the patient and the strength of the sclerosant, to completely alleviate the varicose veins to a satisfactory degree. However, a reduced sclerosant dosage is related to a higher recurrence rate. A multi-centers study by Belcaro et al. reported results from the Venous Disease International Control Trial (VEDICO) based on 749 patients randomly treated by 6 different approaches using measures of occurrence of new varicose veins at 5 and 10 years. Low dose sclerotherapy was less effective than high dose sclerotherapy. At 10 years the occurrence of new veins was 56% for standard sclerotherapy, 49% for high dose sclerotherapy, and 27% for combined surgery and sclerotherapy.

In addition to the injection sclerosant dosage as discussed above, the main issue for the current sclerosing method is that their irritating action can't be predicted. This is because the relatively low viscosity of the sclerosant solution after it is diluted by saline or water to reach the target concentration and also for the ease of injection in the current practice. The dilution of the sclerosant solution with blood occurs immediately upon injection. This dilution effect is less an issue with small vessel due to its smaller blood volume. However, it is difficult to avoid dilution in the large vessel with the current technology. As a result, the original injected concentration is of no real importance. What is important is the diluted concentration of sclerosant at the surface of the endothelium. An injected concentration that is perfectly effective in a smaller vessel may be ineffective in a larger vein simply because dilution reduce the final concentration so low that there is no endothelium injury. The result is that the vein is sclerosed only in the vicinity of the injection. This problem can't be solved by injecting a more potent solution of sclerosing agent because the sclerosing agent may become toxic at such a concentration. It will destroy varicose vein endothelium and may flow into adjacent normal vessels and cause thrombosis of veins, venules, and capillaries. As a result, a well-controlled sclerosant concentration is very important.

Another issue with the current sclerotherapy is that generally it can't be applied to the saphenous vein in the upper thigh region due to the risk of sclerosing the deep veins. When an injection of sclerosant at a high initial concentration is made directly into a perforating vessel, the sclerosant flows into the deep vein. Although the dilution within the deep vein can reduce the chance, it can still happen and cause endothelial injury in the deep system. This overflow of sclerosant can lead to deep vein valve damage and chronic venous insufficiency, to deep vein thrombosis, and to life-threatening pulmonary embolism. This poor control of the flow of the sclerosing agent is worsening by the relatively low viscosity of sclerosing agent in the current sclerotherapy. Many incidences in the saphenous or deep veins have been reported.

### New Sclerotherapy Methods

Some efforts were made to control the concentration of the sclerosing agent in the vein to be treated. A method described in a patent application (US2003/0120217 A1) including a device to flush the varicose veins with saline solution before introducing the sclerosing agent. Another method (US Patent Application 2003/0120256 A1) describes sclerosing the wall of a varicose vein with a catheter with a balloon at the distal end. Then sclerosing agent is used to fill the vein from the distal end of the catheter. However, the efficacy of neither method was documented.

In an effort to enhance the efficacy of sclerothapy, two sclerosants (sodium salicylate and glycerol) were combined in the solution (EP 0,572,963B1). Sodium salicylate is a hyperosmotic sclerosing agent, and glycerol is a detergent sclerosant. A synergetic effect was found while two sclerosants were used together. Less side effects and iatrogenic injury, such as thrombosis, pigmentation, and skin necrosis was reported. However, the concentration of the injection is still diluted in the blood and not in control. Two different sclerosant concentrations were recommended for veins of various sizes.

Foam sclerotherapy represents another effort to control the sclerosant. In this treatment (U.S. Pat. No. 5,676,962; U.S. Pat. No. 6,572,873B1 and US RE38,919), sclerosant solutions are transformed into special foams by forcing gas into the solution for traditional sclerotherapy technique. Polidocanol (POL) is a surfactant and the most commonly used sclerosant to create foams. The foams displace the blood contained in the vein and provide contact of the sclerosing agent with the vascular endothelium. Since the sclerosant is not diluted to the same level by the blood as the conventional sclerotherapy can do, the foams are believed to maintain the predetermined known concentration. It is also believed that the foams are stronger than the sclerosant solutions and to react with endothelium longer at the veins. However, gas (air, CO₂, etc.) is used to form the foams, and there is concern about the solubility of the gas in the body. The unsolved foams may flow to artery and other organs and cause gas embolism. If the patient has a patent foramen ovale, the foams can travel to the brain and may cause serious side effects such as stroke (American venous forum, annual meeting 2006). Autopsy studies show a 27% prevalence of patent foramen ovale in the U.S. However, because it is difficult to detect it with contrast echocardiography, about half of those patients with patent foramen ovale did not know they have this disease. This creates a serious concern for clinicians who practice foam sclerotherapy. In addition, because the foams are lighter weight than the blood, there is also a concern about the ability for the foams to replace blood in the varices to be treated. It is not recommended in the large veins where a higher blood flow is expected. Recently, a synergetic effect was found when Sodium Tetradecyl Sulfate (STS) and Polidocanol (POL) were used together (EP 1,688,135A1) to form foams in the foams sclerotherapy. In this patent application, glycerol and hydroxyethyl starch (HES) were used to modify the viscosity and stability of the sclerosants foams (STS and POL). It is believed that glycerol and HES can increase the consistence of the foams and the contact time between the foams and the endothelium. However, sclerosants foams are created by agitation and have the same gas embolism, light weight and patent foramen ovale concerns as discussed above. In addition, pain and cramping were common complaint from patients when glycerol was used in the sclerotherapy (Alberta Heritage Foundation for Medical Research, TN40, October 2003, p. 25). Hydroxyethyl starch solution is suspected to have detrimental effects on haemostatic mechanisms (Acta Anaesthesiol Scand. 1998 October; 42(9):1104-9) which may compromise blood coagulation, thus increasing the risk of bleeding. As a result, there is a need to improve the performance of sclerotherapy and address the issues raised by foams sclerotherapy. It is desirable to provide a new method that does not need to create foams and thus avoid the complications associated with foams sclerotherapy. This method should treat vessel wall with an injectable sclerosant compound that does not dilute in the blood. This method should also be effective in treating large veins without the concern of deep vein thrombosis.

### SUMMARY OF THE INVENTION

This invention is about a new treatment method for varicose vein, hemorrhoids, venous insufficiencies, esophageal varices, venous-drainage-impotence of the penis, vascular malformation, and excessive blood supplied to tumors. This new method is performed by using a biocompatible thickener to deliver sclerosant to the target vessel wall effectively without the need to form foams. The new thickener/sclerosant compound has a relatively higher viscosity than the blood. It can replace blood in the vessel while injected and form a “plug” in the vessel to temporarily block the blood flow. This invention has several advantages over the “foams” sclerotherapy. First, this new invention doesn't need to use foams. The concern about gas embolism and patent foramen ovale is eliminated. Second, this sclerosant/thickener “plug” is stronger than the foams and stays in the vessel longer than the foams. Due to the enhanced strength and the reduced dilution, the vessel wall is exposed to a controlled concentration of sclerosant during the treatment. Also because of this reduced dilution, a lower concentration of sclerosant is possible in this new therapy. The increased contact time between the sclerosant and the vessel wall will cause more injury to the endothelium and further enhance the efficiency of sclerosant and reduce the sclerosant concentration required. Third, because the strength of the “plug” is higher than the foams, the ends of the “plug” usually pick up fluid in the blood and dissolve in the blood slowly and release the sclerosant in it gradually. The released sclerosant is diluted immediately in the blood and become in-effective without damaging other organs or vessel. On the contrary, the foams are weaker and break up in the blood relatively faster than the “plug”. As a result, a cluster of broken up foams with a relatively high sclerosant concentration can flow to other organs (or deep veins) and may cause damage in those organs. Fourth, this invention also enables the treatment of large veins that can't be treated with current or foams sclerotherapy. With the higher strength of the new sclerosant/thickener “plug” in the vessel and temporarily blocking the blood flow, there is less chance for the sclerosant to flow with the blood and cause damage to the deep system or other healthy vessel. Because a lower concentration of sclerosant is required in this new therapy, any overflow of sclerosant is diluted by the blood and becomes in-effective immediately. As a result, the safety and accuracy of this therapy is enhanced significantly, and a large vessel can be treated without the concern of sclerosing deep veins.

Many thickeners with a higher viscosity than blood can be used in this new treatment. In one embodiment of this invention, a biocompatible hydrogel(s) can be used as a thickener. It is mixed with sclerosant and then injected into vessel by either a needle or a catheter. The examples of this embodiment are PVP and gelatin.

In another embodiment of the invention, the thickener is a biocompatible thickening agent. The viscosity of the sclerosant/thickener compound is higher than the blood. It can replace blood after it is injected into the blood vessel through a needle or a catheter. The examples of this embodiment are dextran, cellulose and cellulose derivatives.

In another embodiment of this invention, the thickener is an environmentally sensitive hydrogel (ESH). The viscosity of the sclerosant/ESH compound increases significantly when it is injected into the blood vessel through a needle or a catheter. The example of this embodiment is polyacrylic acid.

In another embodiment of this invention, the thickener is a thermally reversible hydrogel. It is mixed with sclerosant and then injected into vessel by either a needle or a catheter. A gel (with sclerosant in it) is formed when the thermally reversible hydrogel is in contact with blood. The example of this embodiment is PEO-PPO-PEO block copolymer.

In another embodiment of the invention, the thickener is a self-assembly polymer. The viscosity of the sclerosant/thickener compound increases significantly when it is injected into the blood vessel through a needle or a catheter and reacts with blood. The solubility of the sclerosant decreases significantly after the gelling and prolongs the treatment time on the vessel wall.

In another embodiment of this invention, the thickener is one of the polymer components before polymerization, gelling or crosslinking. The sclerosant/thickener compound and the other polymer component (or catalyst, crosslinking agent, etc.) are introduced to the same vessel either with the same needle or from a separate needle or catheter. The sclerosant/polymer components are controlled to meet and polymerize (or gel or crosslink) at the target site of the vessel. The viscosity of the sclerosant/thickener compound increases significantly after the polymerization (or gel or crosslink). The example of this invention is Alginate (with Ca⁺² as crosslinking agent).

In another embodiment of this invention, the thickener is one of the polymer components before polymerization, gelling or crosslinking. The sclerosant/thickener compound and the other polymer component are introduced to the same vessel from separate lumen of a multi-lumens needle or catheter. They are controlled to meet and polymerize (or gel or crosslink) at the target site of the vessel. The viscosity of the sclerosant/polymer increases after the polymerization (or gel or crosslink). The example of this invention is Alginate (with Ca⁺²).

In another embodiment of the invention, the sclerosant/thickener compound can be used to treat hemorrhoids, venous insufficiencies, varicose vein, esophageal varices, venous-drainage-impotence of the penis, vascular malformation, and excessive blood supplied to tumors. For example, in case of esophageal varice, the sclerosant/thickener compound can be endoscopically injected into the varice to sclerosis the vessel.

Other embodiments encompass the injectables disclosed herein in a ready for use prefilled sterile syringe; in a vial in the form of solution; and in a two compartments prefilled syringe, wherein one compartment contains a curing agent and the other compartment contains a pharmaceutically acceptable thickener and/or sclerosant. Once the sclerosant/thickener compound has been prepared by any one of the existing processes, it can be introduced in any of the previously disclosed methods that can be used later for injecting in the vessels to be treated. Or the sclerosant can be mixed with the thickener right before the injection. The compound disclosed herein may be optionally be sterilized by Gamma, E-beam irradiation, filtering, heating or exposure to ethylene oxide gas.

In another embodiment of the invention, a method is provided for the treatment of varicose vein, comprising the step of administering to a subject an effective amount of sclerosant(s) and one or more thickeners, as a compound which sclerosis blood vessel walls.

### DETAILED DESCRIPTION OF THE INVENTION

This invention is about a new treatment method for hemorrhoids, venous insufficiencies, varicose vein, esophageal varices, venous-drainage-impotence of the penis, vascular malformation, and excessive blood supplied to tumors. The present invention solves many of the problems inherent in the art by providing a biocompatible, injectable thickener to increase the viscosity of the sclerosant/thickener compound without the need to form foams when the compound is injected into the blood vessel. Aspects of the present invention encompass a new biocompatible, injectable non-foaming sclerosant/thickener compound intended for use in sclerotherapy to irritate, dehydrate, change the surface tension of or destroy the endothelium cells. This causes a localized inflammatory reaction and produces small thrombosis that eventually results in permanent fibrosis and elimination of the vein.

The new thickener/sclerosant compound has a relatively higher viscosity than the blood. Without the gas bubbles in it, it is stronger than the foams and can replace blood in the vessel more effectively while injected. As a result, the dilution and diffusion of the sclerosant in the blood is reduced, and the vessel wall is exposed to a controlled concentration of sclerosant during the treatment. Due to this reduced dilution compared with current method, a lower concentration of injected sclerosant can be use in this new therapy. Because of the relatively lower solubility of the thickener/sclerosant compound in the blood, the contact time between the sclerosant and the vessel wall is increased. More injury to the endothelium is expected by the longer exposure to the sclerosant. This can enhance the efficiency of sclerosant and further reduce the concentration of the sclerosant required in this treatment.

Viscosity is a property of fluid related to the internal friction of adjacent fluid layers sliding past one another as well as the friction generated between the fluid and the wall of the vessel. The viscosity of a fluid is basically a measure of how sticky it is. The viscosity of water is about 1 cPs (or 1×10⁻³ Pa s) at room temperature. Blood viscosity is a complicated function of hematocrit, shear rate, and blood composition. Whole blood has a viscosity of 3 to 4 cPs (or 3 to 4×10⁻³ Pa s) depending upon hematocrit, temperature and flow rate.

The equation that governs fluid flowing through a pipe or tube is known as Poiseuille's equation. It is only valid for streamline flow in the tube. Although blood flow through blood vessels in the human body isn't exactly streamline, but applying Poiseuille's equation in this situation is a reasonable first approximation.

Q=πR⁴(P1−P2)/(8 μL)

Where Q is the volume rate of the flow, R is the radius, (P1-P2) is the pressure difference between the ends of the tube, L is the length of the tube, and μ is the coefficient of viscosity. The most interesting thing to notice about the volume rate of flow is that it's inversely proportional to the viscosity. A fluid with a higher viscosity has a lower flow rate. In a blood vessel with both fluids, the fluid with a lower viscosity has a higher flow rate and will leave the fluid with a higher viscosity behind. Eventually, the blood vessel will be filled with a fluid with a higher viscosity. This is the reason why we would like to use the thickener to increase the viscosity of the sclerosant. The sclerosant/thickener compound can replace the blood in the vessel when its viscosity is higher than the blood. In this invention, the viscosity of the sclerosant/thickener is higher than 4×10⁻³ Pa s and preferably higher than 6×10⁻³ Pa s. Poiseuille's equation can also be used to explain the issues with current sclerotherapy. For a typical sclerosant, it is usually diluted by saline or water to reach the target concentration and for the ease of injection through a needle. The viscosity of the resulting solution is usually lower than the blood (typically around 2×10⁻³ Pa s). This solution has a higher flow rate than the blood and is more likely to be diluted by the blood right after the injection and released the sclerosant in the blood. As a result, it is easy to understand why dilution is a problem with the current sclerotherapy.

The present invention solves many of the problems inherent in the art. For example, the sclerosant/thickener compound is stronger than the foams and can displace the blood more effectively and is not diluted until it is dissolved or bio-resorbed. Compared with the lighter weight foams, the thickener is less likely to move with the blood flow, and thus the contact time between the sclerosant and the vessel wall is better controlled. This enhanced control in both concentration and treatment time can significantly reduce complications due to the uncertainties in injecting during the conventional sclerotherapy or foam sclerotherapy as mentioned above. Because the sclerosant is embedded in the thickener, treated zone is the vessel length filled with sclerosant/thickener. The sclerosant concentration of the ‘run-off’ sclerosant outside the treated zone or in the deep vein comes out slowly and is diluted to an in-effective concentration. This will enhance the safety and controllability of the sclerotherapy and reduce the chance for vessel injury outside the target area or deep vein thrombosis. On the contrary, the scleroant foams are weaker than the sclerosant/thickener and tend to break up easily. The “broken up” foams clusters still have a relatively high local sclerosant concentration and may flow to other organs (or deep veins) and cause damage to them.

This invention also enables the use of sclerotherapy for large vessels which can not be treated by foams sclerotherapy. Because the sclerosant/thickener is stronger than the foams and not diluted in the blood and maintains a close contact with blood vessel, potentially less sclerosant is needed to treat varices. In addition, the dissolving (or resorbing) rate of the sclerosant/thickener can be controlled to achieve the optimum treatment time without the issue of sclerosant ‘run-off’. More injury to the endothelium is expected by the longer exposure to the sclerosant/thickener. This can enhance the efficiency of sclerosant and further reduce the concentration of the sclerosant required in this treatment. As a result, the incidence and severity of complications that are created by the high dose of sclerosant used in the current foams sclerotherapy can be reduced. Due to the enhanced safety and controllability with the new invention, large varices can be treated with this new sclerosant/thickener without the issue of sclerosing the deep veins.

### Sclerosants

Sclerosants can be classified into detergents, chemical irritants, and osmotic agents. Their mechanisms to produce endothelial injury vary with these agents. They also vary in their sclerosing power, concentration, the pain provoked on injection and the incidence of other adverse effects. Although a variety of sclerosants are currently available, the ideal agent is yet to be developed. There is no consensus regarding the use of a specific sclerosant, its concentration of dose and for which specific type of varicose veins (part of the reason for this is the lack of sclerosant control during the therapy as mentioned before). The concentration and injection volume of the sclerosants also depends on the interval between injections and whether it is the first injection or re-injection. The most commonly used sclerosing agents are sodium tetradecyl sulfate (STS), sodium salicylate, salicylic acid, hydroxy-polyethoxy-dodecane, hypertonic saline, polidocanol (POL), chromated glycerine, alkali metal tetradecyl sulfate, ethyl alcohol, invert sugar, ethanolamine oleate, sodium morrhuate, polyiodinated solution, Polyiodine iodine, polydodecane, iodic solutions, non-necrotic fatty acid compound (such as sodium oleate, sodium psylliate, sodium ricinoleate, ethylamine oleate, monoethanolamine oleate, sodium formate, sodium acetate and calcium propionate), tetracycline, ferric chloride, quinine, urea, or the combination of the above. The following table lists the concentration of some typical sclerosant used in current sclerotherapy when water or saline (low viscosity) is used as carrier.

TABLE 1
CONCENTRATION OF SOME TYPICAL SCLEROSANTS
(without thickener, only low viscosity carrier such as water or saline)

| | Percentage % |
|---|---|
| Hypertonic saline | 20–23.4% |
| Sodium salycilate | 6–30% |
| STS | 0.5–3% |
| Polidocanol | 0.5–5% |

### Thickeners

The preferred thickener in this invention should be biocompatible. When it is mixed with sclerosant, the compound should have a higher viscosity than the blood and a slower blood solubility rate at 37° C. As a result, the sclerosant/thickener compound is able to form a plug in the vessel after injection with a needle or a catheter and replace the blood in the treated vessel without being diluted before it is dissolved eventually. Many factors affect the viscosity of the thickener. They are the concentration of the thickener, solubility in the blood, molecular weight, temperature, crosslinking agent, process condition, the type and amount of additives and sclerosant in the thickener, etc. The other requirement for sclerosant/thickener is that it should be a fluid with viscosity lower enough before it contacts with the blood so that it can be injected through a needle or a catheter. The sclerosant/thickener should be able to inject at a sufficient rate to fill the vascular lumen before being carried away by blood flow. Once injected in the vessel, the thickener should maintain its properties until it is diluted by the blood. Lastly, it is desirable to have a thickener that can be dissolved or resorbed in the blood after the treatment, because a relatively thickener free vessel is needed after sclerotherapy for the fibrosis to occur on the vessel wall.

The injectable thickeners disclosed herein are biocompatible and preferably biodegradable. The thickener is preferable substantially free of impurity and preferable employed in a highly purified form. They do not cause toxic or inflammatory effects and substantially, if not completely, are degraded, excreted or metabolized by the body. This process may typically take up to several days and is regulated by variables in the formulation and manufacturing of the injectable thickener disclosed herein. The degradation by-products may be mainly expelled via normal respiration and excretion. Many thickener can be used for this application. The following sections will discussed the materials in details.

### Thickening Agents

Viscosity of the compositions may be maintained at the selected level using a pharmaceutically acceptable thickening agent. The thickeners including Acacia, Agar, Alamic Acid, Alginic Acid, Aluminum Monostearate, Attapulgite, Activated Attapulgite, Carbomer Copolymer and Homopolymer, Carbomer Interpolymer, Carboxymethylcellulose (CMC), Carboxymethylcellulose Calcium, Carboxymethylcellulose Sodium, Carrageenan, Cellulose Microcrystalline, Dextrin, Gelatin, Gellan Gum, Guar Gum, Hydroxypropyl Methylcellulose, Hypromellose, Maltodextrin, Methylcellulose, Pectin, Polyethylene Oxide, Povidone, Propylene Glycol Alginate, Colloidal Silicon Dioxide, Sodium Alginate, Tragacanth, Xanthan Gum, Polyacrylic acid, Polyethylene glycol(PEG), lecithin, tridobenzene derivatives, iohexol, iopamidol, iopentol, glycogen, acetyl starch, sucrose, glucose, mannitol, saccharin sodium, dextran, dextrose, sorbitol, phospholipids, cephalin, acetylenic diol, albumin, cellulose derivatives, ethylcellulose, alkyl cellulose, alkoxy cellulose, polyorgano sulfonic acid, and alkoxylated surfactants and their mixtures. The alkoxylated surfactant include, but are not limited to, the group consisting of alkylphenol ethoxylates, ethoxylated fatty acids, alcohol ethoxylates, alcohol alkoxylates, and ethylene oxide-propylene oxide copolymers (Poloxamers, Tetronics or Pluronics). Albumin, Poloxamers, Pluronics, dextran, gelatins and acetyl starch are preferred because they are readily and economically available and are easy to work with. The viscosity of some typical thickeners are listed in Table 2.

TABLE 2
CONCENTRATION and VISCOSITY OF SOME THICKENERS

| | Percentage % | Viscosity cPs |
|---|---|---|
| | Administration | (@25 C.) |
| PVP(Plasdone K12): | 5% | 1 |
| PVP(Plasdone K17): | 5% | 1.5 |
| PVP(Plasdone K90): | 5% | 55 |
| Sodium Alginate(Manuco): | 1% | 120 |
| Sodium Alginate(Kelcosol 100DR): | 1% | 1300 |
| Propylene Glycol Alginate(Kelcoloid): | 1% | 120 |
| Carboxymethylcellulose: | 1% | 25 |

Viscous compositions can be formulated within the appropriate viscosity range to provide longer contact periods with vessel wall. Obviously, the choice of suitable thickener and other additives will depend on the exact method of administration and the nature of the sclerosant. The preferred concentration of the thickener will also depend upon the sclerosant selected. The important point is to use an amount which will achieve the selected viscosity. Viscous compositions are normally prepared from solutions by the addition of such thickening agents. The viscous compositions will typically contain a sufficient amount of a thickening agent so that the viscosity is from about 4 to 100 cPs for needle delivery, although more viscous compositions, even up to 2,000 cPs, may be employed for catheter delivery. It is preferably to have a viscosity of 5 to 600 cPs, since above that range it becomes more difficult to inject. For a high concentration of thickener, less dilution in the blood will occur and less sclerosant is required. However, a less amount of thickener should be used if a small gage needle is used to administer the sclerosant/thickener compound in the vessel. The thickening agent is typically present in a concentration of from about 0.01-80% by weight in the water or other pharmaceutically acceptable carrier, more typically from about 0.05% to about 40%. The sclerosant/thickener compound can be injected into the target vessel through a needle or a catheter. Because the higher viscosity (and lower solubility) of the sclerosant/thickener compound compared with blood, it replaces blood and forms a plug in the vessel. The sclerosant in the compound is then released into the vessel wall or blood stream either by diffusion or by dissolving of the thickening agent(s). The release rate of the sclerosant can be controlled by tailoring the composition, the type of sclerosant and molecular weight of the thickening agent(s).

### Hydrogels

Hydrogels are attractive for different biomedical applications because their favorable biocompatibility. They play an important role in controlled drug delivery because of their pertinence in delivering delicate bioactive agents such as proteins. Hydrogels are network of hydrophilic polymers that can swell in water and hold a large amount of water while maintaining that structure. A three dimensional network is formed by crosslinking polymer chains. The crosslinking can be provided by covalent bonds, hydrogen bonds, van der waals interactions or physical entanglements. Depending on the crosslinking density and molecular weight, the viscosity of the hydrogel can be very high or lower enough to be delivered through the needle or catheter. Examples of some suitable hydrogels for thickeners are polyvinyl pyrrolidone (PVP) polymer or copolymer, polyethylene oxide (PEO) polymer or copolymer, poly(propylene oxide), poly(propylene glycol), poly vinyl alcohol (PVA) polymer or copolymer, Hyaluronic Acid (HA), polyacrylamine, poly(vinylcarboxylic acid), polymethacrylic acid, polyacrylic acid polymer or copolymer, poly amino acids, gel, collagen, fibrin, bioglue, gelatin, alginate, calcium alginate, Cellulose acetate phthalate, cellulosics, Carbopol, Poloxamer, Pluronic, Tetronics, PEO-PPO-PEO triblocks copolymer, tetrafunctional block copolymer of PEO-PPO condensed with ethylenadiamine, polyhema polymer or copolymer, Hypan polymer or copolymer, starch glycolate polymer or copolymer salt, dextran polymer or copolymer, polyoxyalkylene ether, polyvinyl pyridine, polylysine, polyarginine, poly aspartic acid and poly glutamic acid, polytetramethylene oxide, poly(hydroxy ethyl acrylate), poly(hydroxy ethyl methacrylate), hydroxy ethyl cellulose, hydroxy propyl cellulose, methoxylated pectin gels, carrageenan, agar, agarose, oligosaccharide, and macrocyclic polycsaccharide. Alternatively, various hydrogels can be mixed together to tailor the viscosity and the dissolving or resorbing rate in the blood. For example, the PVP can be combined with the Pluronic to increase the dissolving and drug release rate of Pluronic.

The hydrogel can be used as it is or mixed with saline, water, alcohol or other physiological solvents to modify the viscosity of the sclerosant and the dissolving or resorbing rate in the blood. It is mixed with the selected sclerosant and then injected into vessel by either a needle or a catheter. A suitable concentration of the hydrogel will be from 0.01% to 80% based on the total weight and the sclerosant selected. Because the higher viscosity (and lower solubility) of the hydrogel/sclerosant compound compared with blood, it replaces blood and forms a plug in the vessel. The sclerosant in the compound is then released into the vessel wall or blood stream either by diffusion or by dissolving of the hydrogel(s). The release rate of the sclerosant can be controlled by tailoring the composition, crosslinking density, the type of sclerosant and molecular weight of the hydrogel(s). It is known that the release rate of the sclerosant is faster when the sclerosant is relatively small or the hydrogel has a lower crosslinking density. The example of this embodiment is PVP as thickener. It is mixed with sclerosant, saline, water, buffer agent, etc. for a higher viscosity. The preferred viscosity of the final compound is higher than 4×10⁻³ Pa s which is blood viscosity at 37° C. The sclerosant/PVP compound can be injected into the vessel with a needle. Because the sclerosant/PVP compound has a higher viscosity compared with blood, it will form a plug immediately. The sclerosant is then released into the vessel wall through diffusion and as PVP is resorbed into the blood.

Alternatively, some hydrogels require additional curing or crosslinking agent to reach a higher viscosity if it is needed. For those multi-components hydrogels, the sclerosant can mix with component(s) of the hydrogel without causing it to gel. Then, the liquid mixture is injected into the target site to mix with other component(s) of the hydrogel to gel in situ and form a plug of hydrogel/sclerosant. The benefit of this approach is the relatively easier injection through a needle or a catheter due to the lower fluid viscosity of the component(s) before gelling. After the polymerization (or gel or crosslink), the viscosity of the sclerosant/polymer increases, and the solubility of the sclerosant decreases significantly. The example of this embodiment is Ca⁺² Alginate. The Alginate solution can be mixed with a sclerosant (without causing gelling) and be injected into the vessel with a needle or a catheter. Ca⁺² solution is a typical crosslinking agent for Alginate. It can be injected into the target vessel with a needle or a catheter to mix with the Alginate/sclerosant solution and form a gel in the vessel. The resulting sclerosant/Ca⁺² Alginate has a higher viscosity compared with blood and forms a plug immediately. The sclerosant is then released into the vessel wall through diffusion and as Alginate is resorbed into the blood. It is also part of this invention if the sclerosant is mixed with the Ca⁺² and then injected into the vessel to mix with Alginate, which is injected separately.

### Environmentally Sensitive Hydrogels (ESH)

In many situations, hydrogel dissolving in the pharmaceutically acceptable carrier (water, saline) will provide solution viscosity higher than the blood at 37° C. and can be used as thickener for sclerosant. However, it will be ideal to have a sclerosant/thickener formulation with a relatively lower viscosity that can be passed through a needle or catheter for injecting into blood vessels without much effort. After it is in the body, the sclerosant/thickener can change into a higher viscosity material (a gel, etc.) due to the change in temperature, pH, antigen, glucose, or salt content, etc. and replace the blood in the vessel.

Many physical and chemical stimuli have been applied to induce various responses of the environmentally sensitive hydrogels. Physical hydrogels formed with change in temperature, electric field, solvent composition, light, pressure, sound and magnetic field. The chemical stimuli include pH, ions, or salt content, etc. Some of the pH sensitive polymer solutions are: Cellulose acetate phthalate, Carbopol, etc.

### Temperature Sensitive Polymers

The temperature sensitive polymers are part of the ESH and have been studied as potential injectable drug delivery carrier for treating other diseases. They are usually made of polymer with moderate hydrophobic groups or a mixture of hydrophilic and hydrophobic segments. One example is triblock (ABA) copolymers of polyethylene oxide-polypropylene oxide-polyethylene oxide (Poloxamers, Tetronics or Pluronics). They are non-ionic surfactants widely used for medical applications. Some of them have reversible temperature sensitive properties. They form gels at high concentrations and elevated temperatures (higher than the lower critical solution temperature, LCST). At the elevated temperatures, the hydrophobic interaction between the PPO segments of PEO-PPO-PEO triblocks copolymer facilitates the formation of the polymer network while the hydrogen bonding between the polymer and the water becomes weaker. This creates a sudden transition as the hydrated hydrophilic polymer quickly dehydrates and changes to a gels-like structure with a higher viscosity. At a lower temperature, hydrogen bonding between hydrophilic segments of the polymer chain and water molecules dominates and resulting enhanced dissolution and a lower viscosity. This type of thermally reversible polymer can be formulated to allow the injection of a liquid form, through a small catheter or needle at room temperature, which can gel at body temperature.

The LCST of the hydrogel can be altered by changing the polymer composition (hydrophilic/hydrophobic segments ratio and molecular weight). As the polymer chain contains more hydrophobic constituent, LCST becomes lower. Several types of Poloxamers (Poloxamer 188 and 407, Pluronic F127, PF127) have LCST around the body temperature. A preferred Poloxamer according to this invention is Poloxamer 188; a poly(oxyethylene)poly(oxypropylene)copolymer wherein the poly(oxypropylene) portion has an average molecular weight in excess of 12,600 and the polyoxyethylene portion amount to 30% to 70% by weight. The thermo-reversible transition is about 37° C. for a solution of 20% concentration. Another preferred poly(oxyethylene)poly(oxypropylene)copolymer is Pluronic F127. It has an average molecular weight of from about 7500 to about 15,500 with more than 50% oxyethylene units in the total molecular. Its thermo-reversible transition is from about 25° C. to 40° C. for a solution of from 10% to 26% wt in the water. When the thermally reversible hydrogel is used as thickener, it is mixed with sclerosant to form a solution at lower temperature (lower than LCST of the thickener). After the sclerosant/thickener is injected into the blood vessel, it will form a gel immediately and block the blood flow.

In addition to PEO-PPO block copolymers (Poloxamers, Tetronics or Pluronics), their derivatives also process reverse thermo-responsive properties. Cohn, et al. (2003) investigated the bulk polymerization of PEO-PPO-PEO with hexamethylene diisocyanate (HDI) and using phosgene to connect covalently poly(ethylene glycol) and poly(propylene glycol) chains. The new polymers showed reversible thermo-response around 37° C. with much higher viscosities than Pluronic F127. As a result, a lower concentration of the new polymer is required to achieve the same viscosity with F127. The drug release rate in this type of copolymers was slower compared with F127 at the same concentration.

Other than PEO-PPO-PEO triblocks copolymers, the hydrophobic segment (PPO) can be replaced with other hydrophobic polymers and still demonstrate similar thermal reversible properties. Triblock copolymers consisting of poly(ethylene glycol) and other biodegradable polyester blocks, such as poly(L-lactic acid) or poly(glycolic acid), or their co-polyesters were reported to show temperature-dependent reversible sol-gel transitions. Poly(acrylic acid) grafted (PEO-PPO-PEO-PAA) copolymers also have similar transition property and were studied as potential injectable drug delivery vehicles. Many other polymers have similar thermo-responsive phenomenon. They are: graft copolymers of Pluronic and poly(acrylic acid), ethyl(hydroxyethyl) cellulose (EHEC) formulated with ionic surfactants, alkylcellulose, hydroxyalkylcellulose, PEG-PLA-PEG block polymers, Poly(N-isopropylacrylamide)(PNIPAAm), tetrafunctional block copolymer of PEO-PPO-ethylenadiamine, copolymer of PNIPAAm and acrylic acid (AAc), and P(NIPAAm-co-AAc). These solutions have a lower critical solution temperature (LCST), and their viscosity increase while heated above LCST.

### Other Additives

The disclosed solutions in this invention normally contain a major amount of water (preferably purified water, physiological saline, or the like) in addition to the sclerosant and thickener. The compositions can also be lyophilized. Minor amounts of other ingredients such as pH adjusters (e.g., a base such as NaOH), anesthetic agent, buffering agents, and preservatives may also be present depending upon the route of administration and the preparation desired. The compositions can also be isotonic (i.e., it can have the same osmotic pressure as blood).

An aspect of the present invention encompasses an anesthetic to decrease the pain or discomfort associated with injection the compound. Example of anesthetics include but are not limited to lidocaine, xylocaln, novocain, benzocain, prilocaln, ripivacain, propofol, benzyl alcohol, and chlorobutanol. Typically the anesthetic will be used with aqueous base and thus will be mixed with sclerosant/thickener compound prior to administration. A suitable concentration of the anesthetic will be from 0.01% to 6% based on the total weight and the agent selected.

Alternatively, the pH of the sclerosing agent can be modified through a buffer system during the preparation to equal (or close to) the human physiologic pH value. It is believed that the pain the patients feel during injection of sclerosing agent is mainly due to the non-physiologic pH value of the agents. A buffering agent is a chemical compound or compounds that are added to the solution to allow that solution to resist changes in pH as a result of either dilution or small addition of acid or base. A buffering agent can be added in the sclerosant/thickener to adjust the pH value of the compound. Example of buffers are dibasic and monobasic phosphates, citrates, disodium phosphate, sodium diphosphate, disodium hydrogen phosphate and sodium dihydrogen phosphate, sodium phosphate, secondary sodium phosphate, sodium carbonate, phospate buffered saline (PBS), Tris-HCl, citrate-phosphate, Tricine, Hepes and maleate or the salt of weak organic acid with a strong base. The buffering agent, if present, may typically be present in the activated form in a concentration of about 0-4% of the compound. Alternatively, a contrast agent can be added in the carrier for enhanced Fluoroscopy visibility. If desired, isotonicity of this invention may be accomplished using sodium chloride, or other pharmaceutically acceptable agents such as dextrose, boric acid, sodium tartrate, propylene glycol or other inorganic or organic solutes.

A pharmaceutically acceptable preservative can be employed to increase the shelf-life of the compositions. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight and the agent selected.

### Sclerosant Release Rate

After the sclerosant/thickener is injected into the vessel and forms a plug, the sclerosant will be released from the sclerosant/thickener compound in two ways: dissolving and diffusion. First, the thickener begins to draw water from the surroundings and dissolved in the blood at the interface between the blood and the sclerosant/thickener. The sclerosant in the thickener is then released as the thickener dissolves in the blood which is a zero-order process. As a result, the vessel wall is sclerosed starting at the ends of the treated vessel segment and slowly moves to the middle as the sclerosant/thickener dissolving. The sclerosant/thickener dissolving rate is determined by the molecular weight of the thickener, sclerosant/thickener interaction, the type of sclerosant, and additive used, etc. A study done by B. C. Anderson, et al. (2001) indicates that the rate of Pluronic F127 dissolution controls the drug release for hydrophobic small molecular weight drugs.

The second mechanism for the release of sclerosant from the thickener is diffusion. The sclerosant diffuses into the vessel wall at the sclerosant/thickener and vessel wall interface. A study done by S. Mallapragada (1999) indicated that about 70-80% of the drug is released by the diffusion in Pluronic. This is especially true for small size non-surfactant sclerosants. They can easily diffuse into the vessel wall before the sclerosant/thickener is dissolved in the blood. As a result, the sclerosing rate in the vessel wall is controlled by both the sclerosant/thickener dissolving and sclerosant diffusion rates in the thickener.

Some studies were done on the parameters to affect the drug release rate in Pluronic hydrogel. Increasing Pluronic F127 concentration in the gel decreases gel dissolution and drug release rates. The drug release rate was zero-order with either hydrophilic or hydrophobic drugs, and it can also be controlled by the additive in the Pluronic. T. Moore, et al. (2000) studied the dissolution of Pluronic F127 under stirred conditions. The addition of the inorganic salt can increase the bulk viscosity of the gel, however it has no significant effect in the dissolution rate or drug release rate. A study done by Desai et al. (1998) found that additional PEG and PVP increase the dissolving and drug release rate of Pluronic F127 (or PF127). On the other hand, methylcellulose (MC) and hydroxypropyl methylcellulose (HPMC) reduce the dissolving and drug release rate of Pluronic. L. Zhang, et al. (2002) found polyvinyl pyrrolidone (PVP), carboxy methylcellulose (CMC) decreased the release rate of ceftiofur in the Poloxamer 407 gel. As a result, the drug release rate and dissolving rate of sclerosant/thickener can be controlled depending on the concentration, additive and composition of the thickener used.

### Compound Delivery Method

The invention disclosed herein may be used to treat vascular diseases such as hemorrhoids, venous insufficiencies, varicose vein, esophageal varices, venous-drainage-impotence of the penis, vascular malformation, and to stop excessive blood supplied to tumors. In these treatments, the injectable sclerosant/thickener compound is typically introduced into the vessel to be treated by subcutaneous needle injection or by a catheter. For the treatment of venous insufficiencies, the target injection site is superficial veins. The injected sclerosant/thickener compound replaces the blood in the vessel and cause irreversible endothelial injury in the target vein without causing any adverse effects. This causes a localized inflammatory reaction and produces small thrombosis that eventually results in permanent fibrosis and elimination of the vessel.

The injectable sclerosant/thickener compound disclosed herein can be in a ready for use prefilled sterile syringe. Or, it can be provided in a vial in the form of sclerosant/thickener solution. In these embodiments, the end user could add water or other pharmaceutically acceptable carrier and/or additional components prior to injection. Alternatively it can be in a two compartments pre-filled syringe, wherein one compartment contains a sclerosant/thickener compound and the other compartment contains a pharmaceutically acceptable curing or cross linking agent such as CaCl₂ solution if Agnate is used as thickener. Once the sclerosant/thickener compound has been prepared by any one of the existing processes, it can be applied by subcutaneous injection into the vessels to be treated. The compound disclosed herein may be optionally be sterilized by Gamma or E-beam irradiation, filtering, heating or exposure to ethylene oxide gas.

Several other methods can also be used to introduce the new compound in the target vessel. In one embodiment of the invention, sclerosant is mixed with a thickening agent. The solution is injected into the blood vessel through a needle or a catheter. The viscosity of the sclerosant/thickener is higher than the blood and form a plug in the blood vessel. The examples of this embodiment are cellulose and cellulose derivatives.

In another embodiment of this invention, a biocompatible hydrogel is used as thickener. It is mixed with a sclerosant, and then the sclerosant/thickener compound is injected into vessel by either a needle or a catheter. The example of this embodiment is PVP, gelatin and PEO-PPO-PEO copolymers.

In another embodiment of this invention, sclerosant is mixed with one of the polymer components before polymerization, gelling or crosslinking. The sclerosant/polymer component and the other polymer component (or catalyst, crosslinking agent, etc.) are introduced to the same vessel either with the same needle or from a separate needle or catheter. The sclerosant/polymer components are controlled to meet and polymerize (or gel or crosslink) at the target site of the vessel. The viscosity of the sclerosant/polymer increases, and the solubility of the sclerosant decreases significantly after the polymerization (or gel or crosslink). The example of this embodiment is Alginate. The Alginate solution can be mixed with a sclerosant (without causing gelling) and be injected into the vessel with a needle or a catheter. Ca⁺² solution is a typical crosslinking agent for Alginate. It can be injected into the target vessel with a needle or a catheter to mix with the Alginate/sclerosant solution and form a gel in the vessel. The sclerosant is then released into the vessel wall through diffusion and as Alginate is resorbed into the blood. It is also part of this invention if the sclerosant is mixed with the Ca⁺² and then injected into the vessel to mix with Alginate, which is injected separately.

In another embodiment of this invention, sclerosant is mixed with one of the polymer components. The sclerosant/polymer component and the other polymer component are introduced to the same vessel from separate lumen of a multi-lumens needle or catheter. They are controlled to meet and polymerize (or gel or crosslink) at the target site of the vessel. The example of this embodiment is Alginate. The Alginate solution can be mixed with a sclerosant (without causing gelling) and be injected into the vessel through a lumen in a multi-lumens needle or catheter. Ca⁺² solution can be injected into the target vessel through another lumen in a multi-lumens needle or catheter to mix with the Alginate/sclerosant solution and form a gel in the vessel. It is also part of this invention if the sclerosant is mixed with the Ca⁺² and then injected into the vessel to mix with Alginate, which is injected through a different lumen.

In another embodiment of this invention, sclerosant is mixed with an environmentally sensitive hydrogel. The viscosity of the sclerosant/environmentally sensitive hydrogel increases significantly when it is injected into the blood vessel through a needle or a catheter. The solubility of the sclerosant decreases significantly after the gelling. The example of this embodiment is PEO-PPO-PEO block copolymer.

### EXAMPLES

The following examples are included to demonstrate preferred embodiments of the invention (for intravenous administration of sterile sclerosant solutions). It should be appreciated by those of skill in the art that the materials disclosed in the examples represent materials to function well in the practice of the invention, and thus can be considered to constitute preferred modes for its practice. However, those of skill in the art should appreciate that many changes can be made in the specific embodiments which are disclosed and still obtain a similar result without departing from the spirit and scope of the invention. The description is intended to illustrate the principles of the present invention and specific modes encompassed thereby.

### Examples 1

CONCENTRATION OF COMPONENTS IN THE
SOLUTION OF THE PRESENT INVENTION

| | |
|---|---|
| STS | 0.5% |
| Pluronic | 20% |
| Xylocain | 1% |
| disodium phosphate | 0.6% |
| Benzalkonium chloride | 0.2% |
| NaOH | 0.03% |
| water | enough to make 100% |

The compositions of this invention are prepared by mixing the ingredients following generally accepted procedures. For example, 5 mg of STS (obtained from Aldrich) is dissolved in 100 ml of distilled water. 200 mg of Pluronic (F127, obtained from BASF) is added into the solution in a blender at room temperature to assist the mixing. After the mixing with Pluronic, 10 mg Xylocaln (obtained from Aldrich), 2 mg Benzalkonium chloride (obtained from Aldrich), and 6 mg disodium phosphate were added into the solution with appropriate mixing in the blender. Finally, the pH adjusters, 0.3 mg NaOH (obtained from Aldrich), is introduced into the solution to retain the desired pH (7.3). The sterility of the solution is ensured by filtering the solution through a standard 0.3 μm filter with a syringe. Instead of the final concentration, a concentrated mixture may be prepared and adjusted to the final concentration and viscosity by the addition of water or a buffer before injection. Compositions can be administered in dosages and by techniques well known to those skilled in the medical arts and the delivery method used for administration (e.g., needle vs. catheter).

### Examples 2

CONCENTRATION OF COMPONENTS IN THE
SOLUTION OF THE PRESENT INVENTION

| | |
|---|---|
| POL | 0.6% |
| PVP | 23% |
| lidocaine | 1.4% |
| sodium diphosphate | 0.6% |
| chlorobutanol | 0.4% |
| water | enough to make 100% |

### Examples 3

CONCENTRATION OF COMPONENTS IN THE
SOLUTION OF THE PRESENT INVENTION

| | |
|---|---|
| Sodium morrhuate | 6.5% |
| Dextran | 30% |
| ripivacain | 1.7% |
| citrates | 0.9% |
| parabens | 0.2% |
| water | enough to make 100% |

1. A non-foaming sclerosant compound to be delivered to a vascular site for sclerosing vascular tissue comprising:
(a) a pharmacologically effective amount of sclerosant, and
(b) a biocompatible thickener.

2. The compound of claim 1, wherein the sclerosant is selected from the group consisting of sodium tetradecyl sulfate (STS), sodium salicylate, salicylic acid, hydroxy-polyethoxy-dodecane, hypertonic saline, polidocanol (POL), alkali metal tetradecyl sulfate, ethyl alcohol, ethanolamine oleate, sodium morrhuate, polyiodinated solution, Polyiodine iodine, polydodecane, iodic solutions, non-necrotic fatty acid compound, sodium oleate, sodium psylliate, sodium ricinoleate, ethylamine oleate, monoethanolamine oleate, sodium formate, sodium acetate, calcium propionate, tetracycline, ferric chloride, quinine, urea, or the combination of the above.

3. The compound of claim 1, wherein the sclerosant is selected from the group consisting of sodium tetradecyl sulfate (STS), sodium salicylate, hypertonic saline, polidocanol (POL), ethyl alcohol, sodium morrhuate, Polyiodine iodine, ethanolamine oleate or the combination of the above.

4. The compound of claim 1, wherein the sclerosant is present in a concentration of from approximately 0.01% to 50% of the total compound.

5. The compound of claim 1, wherein the biocompatible thickener is selected from the group consisting of thickening agent, hydrogel, environmental sensitive hydrogel, and self assembly polymer.

6. The compound of claim 1, wherein the biocompatible thickener is selected from the group consisting of Acacia, Agar, Alamic Acid, Alginic Acid, Aluminum Monostearate, Attapulgite, Activated Attapulgite, Carbomer Copolymer and Homopolymer, Carbomer Interpolymer, Carboxymethylcellulose (CMC), Carboxymethylcellulose Calcium, Carboxymethylcellulose Sodium, Carrageenan, Cellulose Microcrystalline, Dextrin, Gelatin, Gellan Gum, Guar Gum, Hydroxypropyl Methylcellulose, Hypromellose, Maltodextrin, Methylcellulose, Pectin, Polyethylene Oxide, Povidone, Propylene Glycol Alginate, Colloidal Silicon Dioxide, Sodium Alginate, Tragacanth, Xanthan Gum, Polyacrylic acid, Polyethylene glycol, lecithin, tridobenzene derivatives, iohexol, iopamidol, iopentol, glycogen, acetyl starch, sucrose, glucose, mannitol, saccharin sodium, dextran, sorbitol, phospholipids, cephalin, acetylenic diol, albumin, cellulose derivatives, ethylcellulose, alkyl cellulose, alkoxy cellulose, polyorgano sulfonic acid, and alkoxylated surfactants, alkylphenol ethoxylates, ethoxylated fatty acids, alcohol ethoxylates, alcohol alkoxylates, polyvinyl pyrrolidone (PVP) polymer or copolymer, polyethylene oxide (PEO) polymer or copolymer, poly(propylene oxide), poly(propylene glycol), poly vinyl alcohol (PVA) polymer or copolymer, Hyaluronic Acid (HA), polyacrylamine, poly(vinylcarboxylic acid), polymethacrylic acid, polyacrylic acid polymer or copolymer, poly amino acids, gel, collagen, fibrin, bioglue, gelatin, alginate, calcium alginate, Cellulose acetate phthalate, cellulosics, Carbopol, Poloxamer, Pluronic, Tetronics, PEO-PPO-PEO triblocks copolymer, tetrafunctional block copolymer of PEO-PPO condensed with ethylenadiamine, polyhema polymer or copolymer, Hypan polymer or copolymer, starch glycolate polymer or copolymer salt, dextran polymer or copolymer, polyoxyalkylene ether, polyvinyl pyridine, polylysine, polyarginine, poly aspartic acid and poly glutamic acid, polytetramethylene oxide, poly(hydroxy ethyl acrylate), poly(hydroxy ethyl methacrylate), hydroxy ethyl cellulose, hydroxy propyl cellulose, methoxylated pectin gels, carrageenan, agarose, oligosaccharide, and macrocyclic polycsaccharide, Cellulose acetate phthalate, Carbopol, ethyl(hydroxyethyl) cellulose (EHEC), and mixture of the above.

7. The compound of claim 1, wherein the thickener is selected from the group consisting of albumin, dextran, gelatin, polyvinylpyrrolidone, polyacrylamide, polyethylene glycol, Poloxamer, Pluronic, acetyl starch, mannito, polyvinyl alcohol, and mixture of the above.

8. The compound of claim 1, wherein the thickener is in a pharmaceutically acceptable carrier, wherein the pharmaceutically acceptable carrier comprises water, saline, or plasma.

9. The compound of claim 1, wherein the thickener is present in a concentration of approximately 0.01% to 80% of the total compound.

10. The compound of claim 1, wherein the viscosity of the compound is higher than the viscosity of blood at 37° C.

11. The compound of claim 1, wherein the thickener comprises one of the polymer components before polymerization, gelling or crosslinking.

12. The compound of claim 11, wherein the thickener is controlled to meet and polymerize (or gel or crosslink) with curing agent(s) at the target site of the vessel.

13. The compound of claim 5, wherein the environmentally sensitive hydrogel is sensitive to stimuli selected from the group consisting of temperature, pH, electric field, magnetic field, ionic strength, solvent, pressure, or the combination of the above.

14. The compound of claim 1 further comprising a buffering agent selected from the group consisting of dibasic and monobasic phosphates, citrates, disodium phosphate, sodium diphosphate, disodium hydrogen phosphate and sodium dihydrogen phosphate, sodium phosphate, secondary sodium phosphate, sodium carbonate, phospate buffered saline (PBS), Tris-HCl, citrate-phosphate, Tricine, Hepes and maleate, or the salt of weak organic acid with a strong base.

15. The compound of claim 14, wherein the buffering agent is present in a concentration of from approximately 0.0% to 4% of the total compound.

16. The compound of claim 1 farther comprising an anesthetic selected from the group consisting of lidocaine, xylocaln, novocain, benzocain, prilocaln, ripivacain, propofol, benzyl alcohol, and chlorobutanol.

17. The compound of claim 16, wherein the anesthetic is present in a concentration of from approximately 0.0% to 6% of the total compound.

18. The compound of claim 1, wherein said sclerosant composition is administered in an amount effective to cause fibrosis in blood vessel.

19. The compound of claim 1 farther comprising an additive selected from the group consisting of contrast agent, pH adjuster, preservative, isotonizing agent.

20. A pharmaceutical composition for treating a vascular condition requiring injection into the vascular a non-foaming sclerosant compound comprising:
(a). a pharmacologically effective amount of sclerosant; and,
(b). about 0.01% to about 80% by weight of a pharmacologically acceptable thickener selected from the group consisting of albumin, dextran, gelatin, polyvinylpyrrolidone, polyacrylamide, polyethylene glycol, Poloxamer, Pluronic, acetyl starch, mannito, and polyvinyl alcohol; said compound characterized in that its viscosity above 4 cPs at 37° C.

21. The compound of claim 20, wherein the sclerosant is selected from the group consisting of sodium tetradecyl sulfate (STS), sodium salicylate, hypertonic saline, polidocanol (POL), ethyl alcohol, sodium morrhuate, Polyiodine iodine, ethanolamine oleate or the combination of the above.

22. The compound of claim 20 farther comprising an additive selected from the group consisting of buffering agent, anesthetic, pH adjuster, contrast agent, preservative, and isotonizing agent.

23. A pharmaceutical composition for treating varicose vein, hemorrhoids, venous insufficiencies, esophageal varices, venous-drainage-impotence of the penis, vascular malformation, and excessive blood supplied to tumors, requiring injection into the vascular a non-foaming sclerosant compound comprising:
(a). about 0.01% to about 50% by weight of a pharmacologically effective amount of sclerosant selected from the group consisting of sodium tetradecyl sulfate (STS), sodium salicylate, hypertonic saline, polidocanol (POL), ethyl alcohol, sodium morrhuate, Polyiodine iodine, ethanolamine oleate; and,
(b). about 0.01% to about 80% by weight of a pharmacologically acceptable thickener selected from the group consisting of albumin, dextran, gelatin, polyvinylpyrrolidone, polyacrylamide, polyethylene glycol, Poloxamer, Pluronic, acetyl starch, mannito, and polyvinyl alcohol; said compound characterized in that its viscosity above 4 cPs at 37° C.

24. The compound of claim 23 farther comprising an additive selected from the group consisting of buffering agent, anesthetic, pH adjuster, contrast agent, preservative, and isotonizing agent.
